(19) United States
(12) Patent Application Publication
(10) Pub. No.: US 2021/0236366 A1
(43) Pub. Date: Aug. 5, 2021

(54) Personal micro-climate system for bedridden patients

(71) Applicants: David Serrano, Mayaguez, PR (US); Mauricio A. Lizama, San Juan, PR (US)

(72) Inventors: David Serrano, Mayaguez, PR (US); Mauricio A. Lizama, San Juan, PR (US)

(21) Appl. No.: 17/169,380

(22) Filed: Feb. 5, 2021

Related U.S. Application Data
(60) Provisional application No. 62/970,601, filed on Feb. 5, 2020.

(51) Int. Cl. A61G 10/02; A61M 21/00; A61F 7/00

(52) U.S. Cl. CPC A61G 10/023; A61M 21/02; A61F 7/0053; A61M 21/0094

(57) ABSTRACT

The invention provides a personal micro-climate system that reduces the energy requirement to keep a person cool by providing a personal air-conditioned space specially for bed-ridden patients. A dome is provided to create a personal space that just cools the area where the patient is at avoiding wasting energy in cooling the room's floor, ceiling, walls and empty space. The cooling system can be energized through solar power, deep cycle battery packs, small power plant or through a combination thereof. The system allows for the movement of an automatic or semi-automatic bed. A transparent cover is stretched over a frame structure to allow for maximum visibility from the inside out and vice versa. The system allows ease of opening an attendant window for patient care or to help a bedridden patient in an emergency. Breathing tubes, feeding tubes, IV lines, sensors and cables can be fed into the dome through openings between a mattress cover and the micro-climate dome. The personal microclimate system serves as a positive or negative personal isolation chamber provided with high-efficiency particulate air filters and ultraviolet germicidal irradiation systems. An anteroom can be added next to the micro-climate dome and/or a secondary larger structure may be added to enclose the micro-climate dome.

### FIELD OF THE INVENTION

The present invention relates to the field of medical devices. Specifically, the invention relates to a personal air-conditioned enclosure and cooling system for bed-ridden patients.

### BACKGROUND OF THE INVENTION

Air conditioning systems are usually installed in bedrooms to ensure the comfort of a person, particularly during sleep. Maintaining a proper temperature while a person is in bed is crucial to the attainment of a restful sleep. Existing air conditioning systems are intended to cool (or heat) the entire room, not just the area around a person's bed. Thus, large amounts of energy and power are wasted in cooling (or heating) the walls, floors, ceiling and several areas of empty space.

There have been several attempts to create an enclosed personal space around a bed to save energy by cooling a considerable smaller area. Most of the prior art falls into one of three categories: 1) “sleeping-bag-type” enclosures with a cooling system (Patents IN201941013573A, CN204245776U), 2) cooling systems without an enclosure placed directly next to the user (on the bed headboard for example) (Patents US20130031722A1, CN101467828B), or 3) tent-like enclosure with a small size air conditioner system (Patents US20170299207A1, WO2014199261A3, CN102235726A, US20080264461A1, WO2018145407A1). There are some products in the market that follow some these same characteristics: “Close Comfort PC8—Air Conditioner—Energy saver—White Igloo Bed Tent”, “Portable mini tent air conditioner with battery power” (for camping), “Bed tent” (for light reduction, not air conditioning), and “Tupik Single Bed Air Conditioner”. These devices were intended to increase the comfort of a person while in bed. They were not intended for continuous use of a personal air conditioning system, nor for bed-ridden patients, so they do not address the particular needs of this target group.

Many bedridden persons require continuous air conditioning in order to lower the body temperature and humidity to prevent the onset or worsening of pressure ulcers, bacteria and fungus growth. Conditions such as Miliaria (heat rash) thrive in bedridden patients who have no means to lower room temperature. An appropriately cool air-conditioned room reduces both the heat and humidity that create an ideal environment for the reproduction of fungus and bacteria. However, in hot areas the energy and cost required to keep a small room cool 24/7 is enormous and many times prohibitive. Also, natural disasters like Hurricane Maria in Puerto Rico left many parts of the Island with no power for months. If a family owns a typical power generator, they might be able to power an air-conditioner, but only for periods at a time since most power generators are designed for a cycle of 8 to 10 hours of continuous operation (occasionally, not daily) before having to let it rest, cool down and refuel.

The prior art has many deficiencies that need to be addressed. Patent IN201941013573A comprises a sleeping space close to the person's body (as in a sleeping bag). It does not provide unobstructed space around the person. It is intended for regular beds and not designed to allow for the movements of an automatic or semi-automatic bed. This invention is not intended for bedridden patients and it uses a non-transparent cover that does not allow for patient monitoring nor for the patient to see outside. It has no attendant window or quick access system to help a bedridden patient in an emergency. Finally, this invention does not allow for patient transfer systems to be easily used.

Patent CN204245776U is a temperature controlled outdoor sleeping bag. It is not intended to be used in regular beds nor in automatic or semi-automatic beds. The sleeping space close to the person's body (as in a sleeping bag) and does not provide unobstructed space around the person. It is intended for regular beds and is not designed to allow for the movement of an automatic or semi-automatic bed. This invention is not intended for bedridden patients and it uses a non-transparent cover that does not allow for patient monitoring nor for the patient to see outside. It has no attendant window or quick access system to help a bedridden patient in an emergency. Finally, this invention does not allow for patient transfer systems to be easily used.

Patent US20170299207A1 is an invention intended for regular beds; it was not designed to allow for the movement of an automatic or semi-automatic bed. Its fabric enclosure is composed of several layers, including an insect screen, so the enclosure is not transparent. The fabric enclosure drops down from rods “suspended from the ceiling”. The fabric is not stretched over a frame to help visibility to the outside through transparent material. It has overlapping side openings to enter or leave the bed, but such openings are not sealed. The invention is not intended for bedridden patients and it does not allow for patient transfer systems to be easily used.

Patent US20130031722A1 is a bed with an air-conditioning mechanism included in the mattress and headboard. It would not be suitable for an automatic or semi-automatic bed with moving feet and head areas. The device is intended for regular beds; it was not designed to allow for the movements of an automatic or semi-automatic bed or to accommodate the needs of bedridden patients.

Patent WO2014199261A3 is a four-post bed with an integrated air-conditioning mechanism. It is intended for a fixed, traditional bed and would not be suitable for an automatic or semi-automatic bed with moving feet and head areas. It has non-removable posts that would interfere with transfer or access to a patient in emergencies. This invention is not intended for bedridden patients and it uses a non-transparent cover that does not allow for patient monitoring nor for the patient to see outside. In addition, the design is not intended for use with alternate power systems.

Patent CN101467828B is a cooling system that does not have a cover enclosing the air conditioning area. It is intended for regular beds; it was not designed to allow for the movements of an automatic or semi-automatic bed.

Patent CN102235726A comprises an enclosure designed as a fixed structure of the room; it is not an add-on enclosure. This invention is not intended for bedridden patients and it uses a non-transparent cover that does not allow for patient monitoring nor for the patient to see outside. Its design is not intended for automatic or semi-automatic bed. The structure does not have a quick-detach feature to allow access to a patient in emergencies.

Patent US20080264461A1 is a camping tent, not designed to be used indoors or around a regular bed. A regular tent is used so the cover is not transparent to allow visibility from the inside out and vice versa. Its design is not intended for bedridden patients and the structure does not have a quick access or quick-detach feature to allow access to a patient in emergencies. The system was not designed to allow for alternate power systems.

Patent WO2018145407A1 is an enclosed air purifying system, not an air conditioning system. It is intended for regular beds; it was not designed to allow for the movement of an automatic or semi-automatic bed. The system does not allow for patient transfer systems to be easily used. Since it is not intended for bedridden patients, the structure does not have a quick-detach feature or attendant window to allow access to a patient in emergencies. Additionally, the invention was not designed to allow for alternate power systems.

### SUMMARY OF THE PRESENT INVENTION

The system of the present invention reduces the energy requirement to keep a person cool by providing a personal air-conditioned space for bed-ridden patients. Rather than using a big A/C unit to cool down a regular 1,152 ft³ room (12′×12′×8′), a dome creates a personal 54 ft³ space (6′×3′×3′) that just cools the area where the patient is at. Therefore, a much smaller and energy efficient cooling system can be used, since energy is not wasted in cooling the room's floor, ceiling, walls and empty space. Such cooling system can then be energized through solar power, deep cycle battery packs, small power plant or through a combination of these and other methods.

The present invention has several unique characteristics or combination of features that are particularly useful for bed-ridden persons:
The air-conditioned area provides an unobstructed space around the person.
The system is designed to allow for the movement of an automatic or semi-automatic bed (e.g., head raise/lower, feet raise/lower, bed frame raise/lower).
According to an aspect of the invention, the system is designed for bed-ridden patients.
The transparent cover is stretched over the frame structure to allow for maximum visibility from the inside out and vice versa.
The system allows ease of opening the attendant windows for patient care or to help a bedridden patient in an emergency.
The breathing tubes, feeding tubes, IV lines, sensors and cables can be fed into the dome through openings between the mattress cover and the micro-climate dome.
The structure is easy to remove allowing a patient transfer or to help patient during an emergency.
The micro-climate area provided by the system provides cooling, heating, increasing and/or lowering humidity according to a patient need.
The system can be powered by alternate power systems.

The present invention pertains to a personal micro-climate system for bedridden patients. It is a device that uses little energy to cool down a comfortable space around the patient's bed, rather than using high amounts of energy to cool down the floor, ceiling, walls and space around the patient's whole room. It was originally designed as a solution for bedridden patients who were left without power for months in the aftermath of Puerto Rico's hurricane Maria (2017). The lack of power, the scarcity of power plants and of the gasoline to power them, prevented the use of home air conditioners. This elevated room temperature and humidity at patients' homes sharply increased skin issues like pressure ulcers, heat rashes, and bacterial and fungus growth.

The personal micro-climate system is a lightweight tent-like frame structure covered with plastic transparent material to isolate a space comprising a hospital bed mattress (preferably, approximately 6′ long by 3′ wide) and preferably 3′ high above the mattress. The frame structure is designed to allow for the normal movement of an automatic or semi-automatic bed (head raise/lower, feet raise/lower). In the preferred embodiment, the frame is constructed of square aluminum rod bent to form a tent-like shape. There are two arcs, one going from the top-left to the bottom-left side of the bed, and the other going from the top-right to the bottom-right side of the bed. The arcs are parallel to each other and they extend about three feet above the top of the bed's mattress. The arcs are supported by a rectangular structure that surrounds all sides of the bed and attaches to the arcs at its corners. Two parallel rods extend between the top of both arcs in order to keep them parallel and to support the plastic enclosure. The aluminum rods are connected together using machine screws and nuts. A notch at the end of each rod allows for a solid and slip-free attachment.

A plastic cover encloses the space around the patient that will be cooled by the cooling system and serves as a barrier for heat exchange with the surrounding room area. The cover consists of transparent plastic with a top panel that runs from the head side to the feet-side of the bed and two side panels with large attendant windows cut out. The panels are sewn or glued together, leaving “channels” for the structural rods to pass through. Attendant windows on either side of the structure are provided, to facilitate feeding, communication or comfort. A cutout for the attendant windows is made at the side of each panel. The top and both sides of each window are cut, but the bottom is left uncut to allow the windows to open down. A double-sided zipper or Velcro is fixed to all the cut sides of each window, so they can be tightly closed. The enclosed space is not completely sealed, to allow for breathing air to flow in and waste gases to go out. Breathing tubes, feeding tubes, IV lines, sensors and cables can be fed into the dome through openings between the mattress cover and the micro-climate dome. In case of patient transfer or emergency, the dome can be lifted to a side or taken completely off the bed. A small energy-efficient system cools the space inside the dome. The cooling system is located outside the plastic enclosure, in a box placed on the floor or on a table next to the bed. The cooling system has a flexible tube that extends to connect to the plastic enclosure in order to carry the cooled air inside the enclosure. This cooling tube enters the enclosure at the “head-side” of the bed to provide for maximum cooling at the patient's head area. The exit opening for the warm air to come out of the enclosure is also located at the “head-side” of the bed and above the cooling tube opening. A wall plug-in, small power plant, deep cycle batteries, solar power system, or other similar power source could power the system.

For patients that do have access to air-conditioner 24/7, the personal micro-climate system can be stored and kept for emergency use when power goes out or when the regular AC breaks down. For patients who cannot afford having a conventional AC system running year-round, the personal microclimate allows for a cost effective alternative with a much lower energy consumption. Finally, for persons without disabilities, the personal micro-climate system may allow for cost effective cooling during extended power outs (i.e., natural disasters) and non-emergency applications such as cooling baby cribs, camping tents and pet houses.

### BRIEF DESCRIPTION OF THE DRAWINGS

Further features and advantages of the invention will become apparent from the following detailed description taken in conjunction with the accompanying figures showing illustrative embodiments of the invention, in which:

FIG. 1 is a perspective view of an exemplary embodiment of the system according to the present invention.

FIG. 2 is a perspective view of one embodiment of the frame structure according to the present invention. The two inserts show details of the connection system for the rods using perforated notches at each end.

FIG. 3 is a perspective view of an exemplary embodiment according to the present invention. The frame structure is shown placed around a bed, with the transparent cover in place and the person in the bed inside the enclosure. The attendant window is shown in the closed position.

FIG. 4 is a perspective view of an exemplary embodiment according to the present invention. The frame structure is shown placed around a bed, with the transparent cover in place and the person in the bed inside the enclosure. The attendant window is shown in the open position.

FIG. 5 is a perspective view of another exemplary embodiment of the system according to the present invention.

FIG. 6 is an exploded perspective view of the exemplary embodiment of the system of FIG. 5.

FIG. 7 is an exploded top view of the exemplary embodiment of the system of FIG. 5.

FIG. 8 is a top view of a frame of the exemplary embodiment of the system of FIG. 5.

FIG. 9 is a partial bottom view of a bottom covering of the plastic cover according to the present invention.

FIG. 10a shows an embodiment of an anteroom next to the enclosure according to the present invention.

FIG. 10b shows an embodiment of a secondary larger structure enclosing the enclosure according to the present invention.

Throughout the figures, the same reference numbers and characters, unless otherwise stated, are used to denote like elements, components, portions or features of the illustrated embodiments. The subject invention will be described in detail in conjunction with the accompanying figures, in view of the illustrative embodiments.

### DETAILED DESCRIPTION OF THE PREFERRED EMBODIMENTS

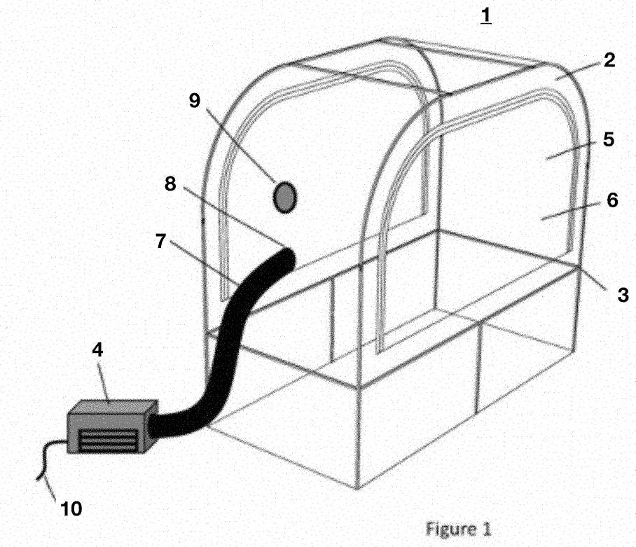

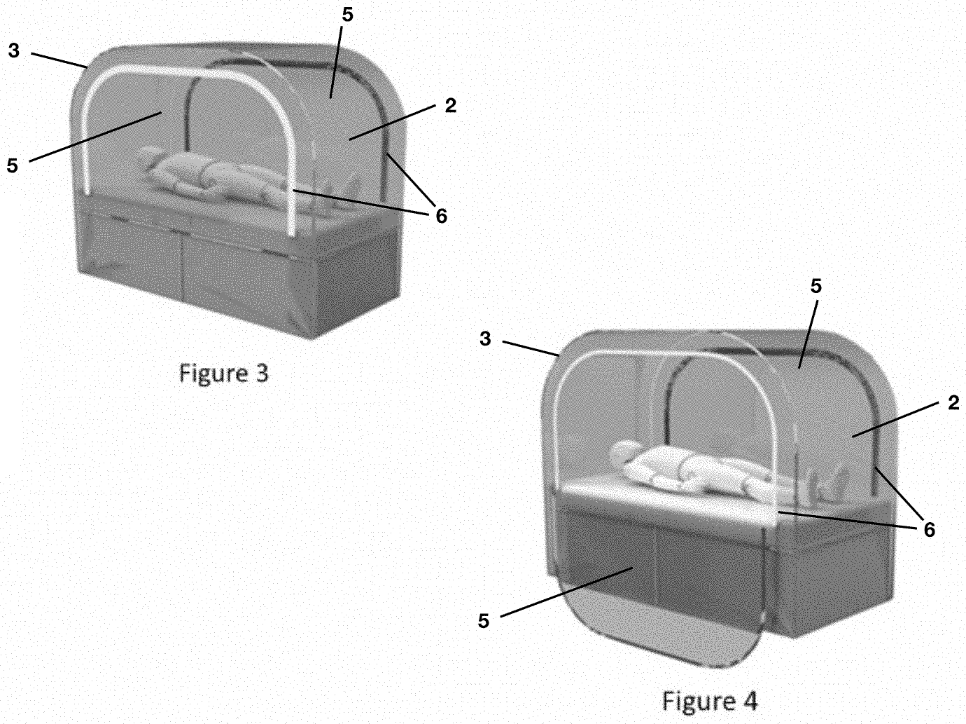

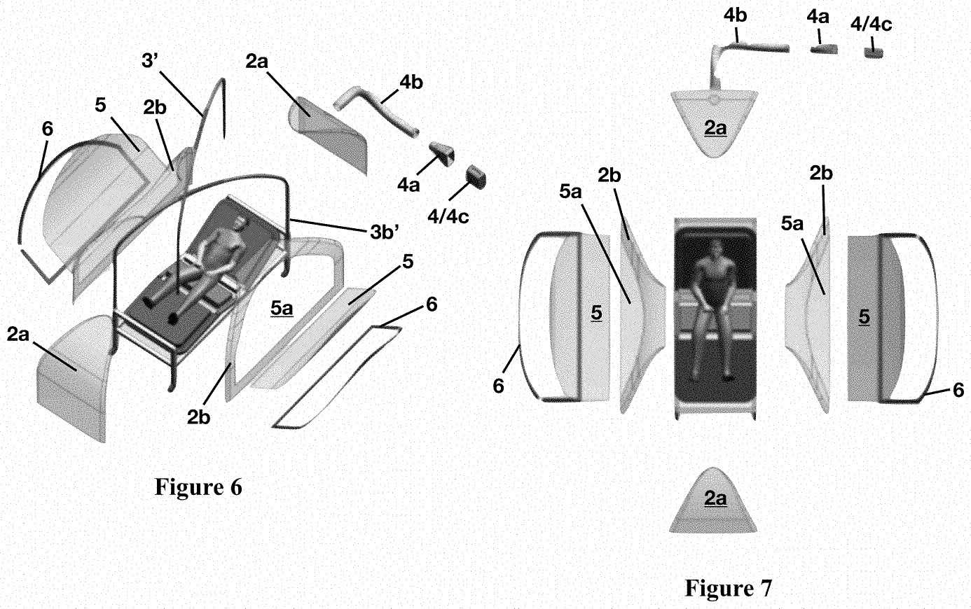

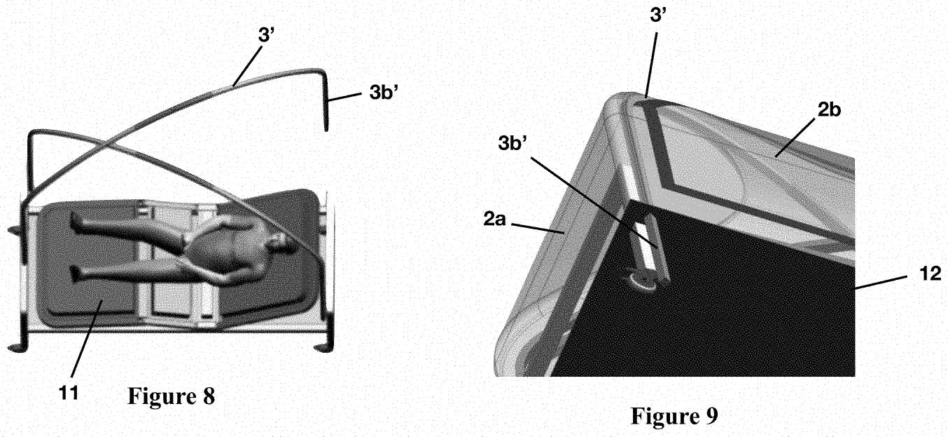

The invention comprises of three main elements: a frame structure, a cover enclosure and a cooling system. The invention along with the several embodiments will be explained in conjunction with FIGS. 1-10b.

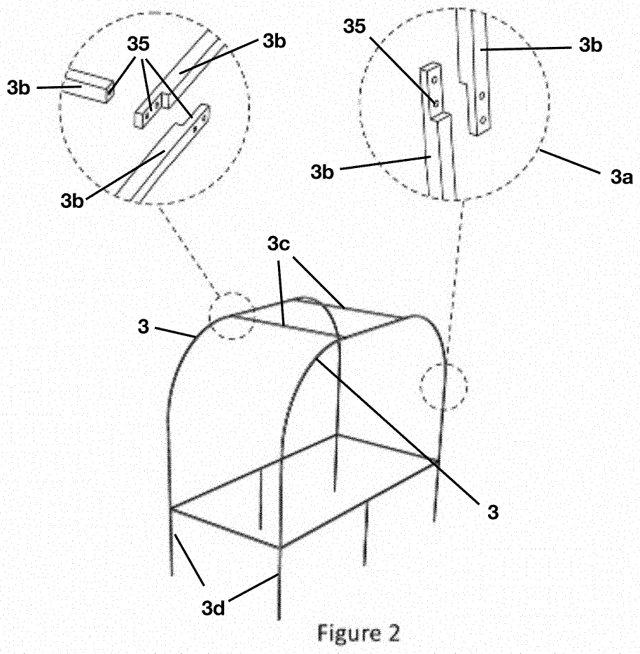

The frame structure is preferably constructed of ⅜″ square aluminum rod (3b). As shown in FIG. 1 and FIG. 2, there are two arcs (3), one going from the top-left to the bottom-left side of the bed (11), and the other going from the top-right to the bottom-right side of the bed (11). The arcs (3) are parallel to each other and they extend preferably about three feet above the top of the bed's mattress. On the rods that make the arcs (3), a notch preferably 2″ long is made at the end of each rod to remove half the width of the rod. Two holes (35) are perforated inside the notches to insert machine screws to connect the rods. The arcs (3) are supported by a rectangular structure (see FIG. 2) that surrounds all sides of the bed and attaches to the arcs at its corners. Two parallel rods (3C) extend between the top of both arcs (3) in order to keep them parallel and to support the plastic enclosure. The rods used for the rectangular structure and the parallel rods used at the top of the structure have perforations (35) at the end of each side, in the center of the square end, parallel to the rod. The frame structure is supported on the floor using the four posts of the arcs.

A plastic cover (2) encloses the space around the patient that will be cooled by the cooling system (4) and serves as a barrier for heat exchange with the surrounding room area. The cover (2) is preferably made of transparent vinyl plastic with a top panel that runs from the head-side to the feet-side of the bed and two side panels with large attendant windows cut out (5). In an embodiment, the panels (2) are sewn together at the edges. However, other means (permanent and/or removable) such as but not limited to adhesive, Velcro, or any combination of permanent and removable elements can be used to attach the panels together.

At the edge between a side panel and a top cover, a channel of preferably ½″ is left in the inside part of the cover so that the frame structure can slide in to secure the cover on the frame. Attendant windows (5) are provided on either side of the structure, to facilitate feeding, communication or comfort. A cutout (5a) for the attendant windows is made at the side of each panel. The top and both sides of each window are cut at preferably 6″ from the edge of the panel and down to the top side of the bed mattress, but the bottom is left uncut to allow the windows to open down. A double-sided closing element (6) such as but not limited to a zipper is fixed to all the cut sides of each window (5), so it can be tightly closed. The enclosed space is not completely sealed, to allow for breathing air to flow in and waste gases to go out. Thus, the plastic cover (2) extends from the top of the frame structure down to the frame of the bed, at the lower part of the mattress; but the bottom part of the plastic cover is not sealed around the bed, so that the enclosure is not air-tight. Breathing tubes, feeding tubes, IV lines, sensors and cables can be fed into the enclosure through openings between the mattress cover and the micro-climate enclosure. In case of patient transfer or emergency, the enclosure can be lifted to a side or taken completely off the bed.

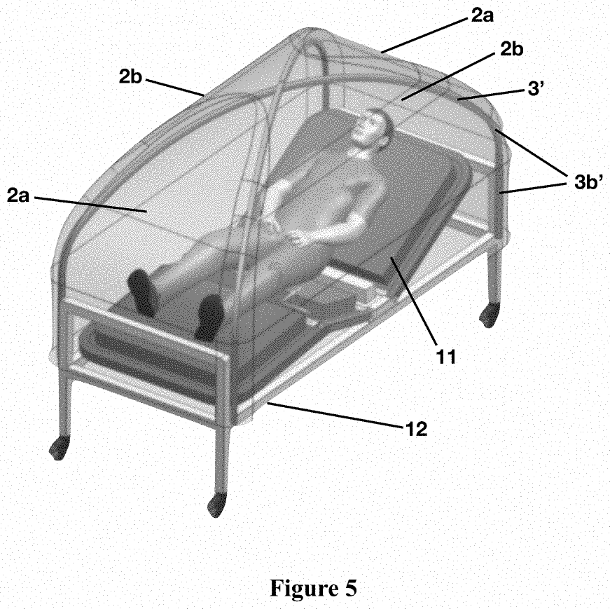

According to another embodiment, there are two arcs (3b′), one going from the top-left to the bottom-right side of the bed, and the other going from the top-right to the bottom-left side of the bed (FIG. 5). The arcs (3b′) cross at the top center of the bed at preferably about three feet above the top of the bed's mattress. At the point where the rods cross, a pin goes through the center of both rods to keep them stacked together and aligned in a center position. This pin also allows the X frame formed by the two arcs (3b′) to be collapsed in a flat position for storage. The frame structure is supported on the floor using the four posts of the arcs (FIG. 2). Like the previous embodiment, attendant windows (5) are provided on either or both sides of the structure, to facilitate feeding, communication or comfort. A cutout (5a) for the attendant windows (5) is made at the side of each panel (2b). The top and both sides of each window are preferably cut at 4″ from the edge of the panel and down to the top side of the bed mattress, but the bottom is left uncut to allow the windows to open down. A double-sided closing element (6) such as but not limited to a zipper is fixed to all the cut sides of each window (5), so it can be tightly closed. The enclosed space is not completely sealed, to allow for breathing air to flow in and waste gases to go out. Thus, the plastic cover (2) extends from the top of the frame structure down to the frame of the bed (11), at the lower part of the mattress; but the bottom part of the plastic cover (2) is not sealed around the bed, so that the enclosure is not airtight. Breathing tubes, feeding tubes, IV lines, sensors and cables can be fed into the enclosure through openings between the mattress and the micro-climate enclosure. In case of patient transfer or emergency, the enclosure can be lifted to a side or taken completely off the bed.

A small portable air conditioner cooling system (4) cools the space inside the dome. The cooling system (4) can be located outside the plastic enclosure (2), in a box placed on the floor next to or under the bed. Alternatively, the cooling system (4) can be located inside the plastic enclosure (2), in a shelf placed on the bed's headboard or footboard. The cooling system (4) has a flexible plastic tube (4b/7) that extends to connect to the enclosure in order to carry the cooled air inside the enclosure. This cooling tube enters the enclosure preferably at the center (7) of the “head-side” of the bed and below the top of the frame (for example, 18″), to provide for maximum cooling at the patient's head area. An exit opening (9) for the warm air to come out of the enclosure is also preferably located at the center of the “head-side” of the bed and above the cooling tube opening (for example 12″). Alternatively, the cooling system has a flexible plastic tube that extends outside of the enclosure for the warm air to exit. The cooling system can also be located outside the enclosure with flexible tubes connected to the enclosure to allow cool air in and warm air out. If necessary, a cooler adapter/manifold is provided to couple the output of the cooling system (4) to the tubing (4b/7).

In an embodiment, the cooling system can be powered (10) by, but not limited to an AC plug (110v or 220V) from a wall outlet, a DC battery system with inverter if necessary, a power generator, or any combinations thereof.

According to another embodiment of the invention, the personal microclimate system of the present serves as a personal isolation chamber for contagious diseases. Due to the recent COVID-19 pandemic, the need for isolation rooms increased exponentially. These rooms are scarce, mostly available at hospitals and hard to equip due to the need to provide a segregated space with either positive or negative air pressure inside depending on the need.

Positive pressure rooms maintain a higher pressure inside the area than that of the surrounding environment. This means air can leave the room without circulating back in. In this way, any airborne particle that originates in the room will be filtered out. Thus, in a contagious disease situation like COVID-19, vulnerable patients would be placed inside a positive pressure room to prevent them from getting the virus. In contrast, patients who are already have the infectious condition would be placed in a negative pressure room to protect people outside the room from exposure. A negative pressure room uses lower air pressure inside the area to allow outside air in while preventing internal air from leaving the space.

The personal microclimate system of the invention serves as a positive or negative personal isolation chamber, depending on setup of the equipment used to direct the air that goes in and out of the enclosure. For a positive pressure isolation chamber, incoming air is filtered through both high-efficiency particulate air filters and ultraviolet germicidal irradiation systems (4c). Air pressure inside the enclosure would be maintained higher than that of the surrounding environment. This setup would prevent a vulnerable patient inside the chamber from being infected by an external pathogen. For a negative pressure isolation chamber, high-efficiency particulate air filters and ultraviolet germicidal irradiation systems (4c) would be used to keep the air pressure inside the chamber lower than that of the surrounding environment. In this setup, an infected patient may be isolated inside the chamber to protect people outside the room from exposure. In order to maintain the required pressure level, a bottom part of the plastic cover is provided with a bottom covering (12) that is permanently or removably attached to the plastic cover (2) as illustrated in FIG. 5 and FIG. 9. In a preferred embodiment, the bottom covering (12) is made from a material such as but not limited to acrylic felt that is durable, resilient and resistant against tearing. This bottom covering (12) can be provided at the floor level or at the mattress level above or below a bed frame. In addition, the bottom covering (12) can have passthrough openings to allow passage of the frame posts (FIG. 9).

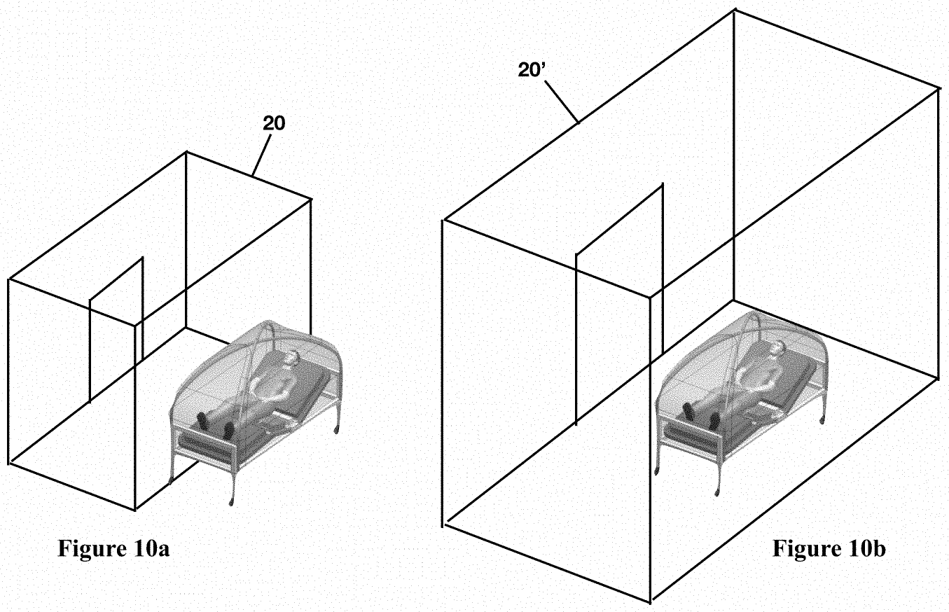

To maintain the appropriate air pressure inside the enclosure while facilitating patient handling and caregiver assistance an anteroom (20) is added next to the enclosure (FIG. 10a). Alternatively, a secondary larger structure (20′) may be added to create an enclosure within an enclosure setup (FIG. 10a). The addition of an anteroom would also facilitate the conversion of a positive pressure chamber to a negative pressure chamber and vice versa. A positive or negative air pressure chamber may be used in places where a controlled air pressure room is not available, viable or cost effective, such as elderly homes, emergency shelters, patient overflow areas or even in private homes.

There are some aspects of the invention that may be substituted for alternative elements. The frame support structure can be composed of inflatable tubes, aluminum, fiberglass or any other material that would support the enclosure and allow for the normal movement of an automatic or semi-automatic bed. The cover may be a single or multiple-layer material, transparent or colored, or a combination of materials in multiple layers and air pockets. A different material might be used for the body area where no transparency may be needed. The attendant windows seals and cover release system may be magnetic, hook & loop, zippered, weighted or any other available material. Cooling may be accomplished by thermoelectric cooling (i.e., Peltier device), a vortex tube, vapor-compression or any other means of conduction, convection, radiation or evaporation as well as any combination thereof. The cooling tube may enter the enclosure at the “feet-side” of the bed or at one of the sides. The exit opening for the warm air to come out of the enclosure may be located in any side of the enclosure. This opening may allow for the air to exit towards the room or it may be connected to a tube that reverts the air to the cooling system.

The present invention has been illustrated by the description of an exemplary processes and system components and while the various processes and components have been described in considerable detail, it has not been the intention of the presentation in any way as to limit the scope of the invention to such details as to preclude any additional advantages and modifications which may also readily appear to those ordinarily skilled in the art. The invention in its broadest aspects is therefore not limited to the specific details, implementations, or illustrative examples shown and described. Accordingly, departures may be made from such details without departing from the spirit or scope of applicant's general inventive concept.

1. A personal micro-climate system for bedridden patients comprising:
a frame having a first arc and a second arc configured to be provided above a bed;
a cover coupled to said frame and including at least one window provided on a side of said cover, wherein said cover further comprises at least one opening; and
a cooling system coupled to said at least one opening via a tube.

2. The personal micro-climate system according to claim 1, wherein said first arc has a first end configured to be positioned at an upper left corner of the bed and a second end configured to be positioned at a lower right corner of the bed; and said second arc has a first end configured to be positioned at an upper right corner of the bed and a second end configured to be positioned at a lower left corner of the bed.

3. The personal micro-climate system according to claim 2, wherein said first arc and said second arc are joined at their middle portions forming an X-shaped frame.

4. The personal micro-climate system according to claim 3, wherein said first arc and said second arc are stacked over each other at said joined middle portion so that both arcs are rotated from a closed position into an open position and vice versa.

5. The personal micro-climate system according to claim 1, wherein said first arc has a first end configured to be positioned at an upper left corner of the bed and a second end configured to be positioned at a lower left corner of the bed; and said second arc has a first end configured to be positioned at an upper right corner of the bed and a second end configured to be positioned at a lower right corner of the bed.

6. The personal micro-climate system according to claim 5, further comprising at least one rod extending between a top portion of said first and second arcs.

7. The personal micro-climate system according to claim 1, wherein each of said first arc and said second arc are formed by plural parts that are attached and combined to form said arcs.

8. The personal micro-climate system according to claim 7, wherein ends of said plural part comprise a notch to provide a slip-free attachment.

9. The personal micro-climate system according to claim 1, further comprising a closing element provided on said side of the cover to selectively open or close said at least one window from inside and outside of said cover.

10. The personal micro-climate system according to claim 1, further comprising a bottom cover attached at a bottom part of said cover to seal the bed inside said cover.

11. The personal micro-climate system according to claim 10, wherein said cooling system comprises positive-pressure system to maintain a positive pressure inside said cover.

12. The personal micro-climate system according to claim 10, wherein said cooling system comprises negative-pressure system to maintain a negative pressure inside said cover.

13. The personal micro-climate system according to claim 1, further comprising an anteroom provided adjacent to said covered bed.

14. The personal micro-climate system according to claim 1, further comprising an enclosing structure larger than said covered bed to enclose said covered bed.
